SEQUENCE LISTING (2 sequences)

SEQ ID NO: 1 - RNA, 22 nt, homo sapiens
uggcagugucuuagcugguugu

SEQ ID NO: 2 - RNA, 22 nt, homo sapiens
ucaccggguguaaaucagcuug

(19) United States
(12) Patent Application Publication
(10) Pub. No.: US 2022/0162702 A1
(43) Pub. Date: May 26, 2022

(54) DIAGNOSIS OF NON-ALCOHOLIC STEATOHEPATITIS

(71) Applicant: GENFIT, LOOS (FR)

(72) Inventor: JOHN BROZEK, SAINT-AMAND-LES-EAUX (FR)

(21) Appl. No.: 17/437,063

(22) PCT Filed: Mar. 12, 2020
(86) PCT No.: PCT/EP2020/056682

§ 371 Date: Sep. 8, 2021

(30) Foreign Application Priority Data
Mar. 13, 2019 (EP) 19305305.5

(51) Int. Cl. C12Q 1/6883; A61K 31/192; A61K 31/426; G01N 33/68

(52) U.S. Cl. CPC C12Q 1/6883; A61K 31/192; A61K 31/426; G01N 33/6893; G01N 2800/52; C12Q 2600/178; C12Q 2600/106; G01N 2800/085; C12Q 2600/158

(57) ABSTRACT

The present invention relates to a non-invasive method for classifying a subject as a potential receiver or non-receiver of a treatment for non-alcoholic steatohepatitis.

### FIELD OF THE INVENTION

The present invention relates to a non-invasive method for classifying a subject as a potential receiver or non-receiver of a treatment for non-alcoholic steatohepatitis.

### BACKGROUND OF THE INVENTION

Non-alcoholic steatohepatitis (NASH) is a progressive disease of the liver characterized histologically by fatty acid accumulation, hepatocyte damage and inflammation resembling alcoholic hepatitis. NASH can lead to liver fibrosis, cirrhosis, liver failure and/or hepatocellular carninoma (HCC). Along with the obesity and type-2 diabetes rates in the world, the incidence of NASH has increased in recent years, and patients who develop NASH have an increased rate of liver-related mortalities. Since the prevalence of these diseases is increasing, the prevalence of NASH is also expected to increase and therefore, NASH has become a worldwide emerging public health issue. These major concerns underscore the need for the development of more sensitive and reliable method for a diagnostic of NASH.

As of today, histological analysis of liver biopsies remains the optimal approach for differentiating NASH from early stage steatosis. However, liver biopsy has a number of obvious drawbacks. First, the material collected in liver biopsy represents only a very small part of the liver of the diagnosed subject, thereby raising doubts on whether the collected sample is representative of the global state of the subject's organ. Moreover, liver biopsy is a very invasive procedure that may be cumbersome, worrisome and painful for the patient, and which raises concerns about morbidity and mortality. At last, in view of the foregoing, liver biopsy cannot be reasonably proposed as a routine procedure for determining whether a person in the general population, or even patients at risk of NASH, suffers from NASH and/or for determining the activity, the stage, or the severity of NASH in said person.

These drawbacks of biopsy-based NASH diagnosis led to an active development of non-invasive methods for the detection of NASH. For example, WO2017046181 and WO2017167934 provide non-invasive diagnosis based on the measure of the level of circulating biomarkers.

The NASH prevalence in the general population has already reached 6-15% in the United States and 3-16% in Europe. There is an obvious discrepancy between the total number of liver biopsies performed each year (about 53,000 in the US) and the estimated number of NASH patients in the general population. This very low diagnosis rate reflects poor disease awareness in the medical community partly because NASH is a silent, slowly progressing, asymptomatic disease with no approved treatment yet, but most importantly because of lack of simple, reliable and widely accessible tests to identify NASH patients at highest risk of developing clinical outcomes, who should be managed and possibly treated for their condition.

Transient elastography (TE) is a non-invasive procedure that is used to examine the stiffness of liver tissue (Friedrich-Rust, 2008). TE is an ultrasound-based technology measuring liver stiffness by the difference in velocity of elastic shear wave propagation across the liver. TE can be carried out e.g. by using the echograph commercialized under the name FIBROSCAN® (EchoSens, Paris, France). However, TE could be influenced by patient-dependent factors, including liver inflammation, liver congestion, and biliary obstruction. Ultrasound-based elastography such as FIBROSCAN® and shear wave elastography has moderate to high accuracy in diagnosing advanced fibrosis or cirrhosis. TE provides a reliable method for detecting cirrhosis and excluding significant fibrosis but F2 fibrosis which is not an advanced fibrosis stage or NASH without fibrosis usually cannot be accurately detected with these techniques.

The present invention relates to an improvement of such non-invasive methods.

### SUMMARY OF THE INVENTION

The present invention relates to a method for the diagnosis of non-alcoholic steatohepatitis (NASH), for the classification of a subject as a receiver or non-receiver of a treatment for NASH, or for monitoring the efficiency of a treatment for NASH, comprising:
i) measuring liver fibrosis of said subject with a physical method; and
ii) measuring the level of at least one circulating marker in a body fluid of said subject, selected in the group consisting of hsa-miR34a, A2M, YKL40 and Hb1Ac.

In a particular embodiment, measure i) comprises measuring liver stiffness of said subject. In a further particular embodiment, liver stiffness is measured by measuring the difference in velocity of elastic shear wave propagation in the liver

In yet another embodiment measure step ii) comprises the measure of the level of at least two circulating markers selected in the group consisting of hsa-miR34a, A2M, YKL40 and Hb1Ac. In a further embodiment, measure ii) comprises the measure of the level of hsa-miR34a and A2M.

According to a particular embodiment, measure ii) comprises the measure of the level of at least three circulating markers selected in the group consisting of hsa-miR34a, A2M, YKL40 and Hb1Ac, such as the level of hsa-miR34a, A2M and YKL40.

In yet another embodiment, measure ii) comprises the measure of the level of hsa-miR34a, A2M, YKL40 and Hb1Ac.

In another embodiment, measures i) and ii) are combined to calculate a score for the diagnosis of non-alcoholic steatohepatitis (NASH), for the classification of a subject as a receiver or non-receiver of a treatment for NASH, or for monitoring the efficiency of a treatment for NASH.

In yet another embodiment, the method is for the classification of a subject potentially having a steatosis score≥1, a hepatocyte ballooning score≥1, a lobular inflammation score≥1, a NAS≥4 and a fibrosis stage≥2.

Another aspect of the invention relates to an anti-NASH compound for use in the treatment of NASH, wherein the subject to be treated as been classified as a receiver of a treatment for NASH according to the method disclosed herein. In a particular embodiment, said anti-NASH compound is elafibranor or a pharmaceutically acceptable salt thereof In yet another embodiment, said anti-NASH compound is nitazoxanide or a pharmaceutically acceptable salt thereof. According to a further embodiment, anti-NASH compound is elafibranor or a pharmaceutically acceptable salt thereof, for use in combination with nitazoxanide or a pharmaceutically acceptable salt thereof.

### DETAILED DESCRIPTION OF THE INVENTION

The present invention relates to a method for the diagnosis of NASH, for the classification of a subject as a receiver or non-receiver of a treatment for NASH, or for monitoring the efficiency of a treatment for NASH. The method of the invention is particularly useful in the diagnosis of fibrosing NASH, in the classification of a subject as a receiver or non-receiver of a treatment for fibrosing NASH, or for monitoring the efficiency of a treatment for fibrosing NASH.

According to the invention, the term “non-alcoholic steatohepatitis” refers to a non-alcoholic fatty liver disease (NAFLD) condition characterized by the concomitant presence of liver steatosis, hepatocyte ballooning and liver inflammation at histological examination, in the absence of excessive alcohol consumption and after excluding other liver diseases like viral hepatitis (HCV, HBV). According to the invention, the term “steatosis” refers to the process describing the abnormal retention of lipids or fat accumulation within the liver. According to the present invention, the term “hepatocellular ballooning” is usually defined, at the light microscopic level, based on hemotoxylin and eosin (H&E) staining, as cellular enlargement 1.5-2 times the normal hepatocyte diameter, with rarefied cytoplasm. It refers more generally to the process of hepatocyte cell death. According to the present invention, the term “lobular inflammation” refers to the presence of lobular inflammatory foci (grouped inflammatory cells) at microscopic examination of a hematoxylin and eosin (H&E) stained slice of a liver biopsy.

According to the present invention, the “NAFLD-Activity score” or “NAS” refers to the sum of steatosis, hepatocellular ballooning, lobular inflammation scores, as follows:
S: Steatosis score: 0: <5%; 1: 5-33%; 2: 34-66% and 3: >66%;
LI: Lobular Inflammation score (foci/x20 field): 0: none; 1: <2; 2: 2-4 and 3: >4;
HB: Ballooning degeneration score: 0: none; 1: few; 2: many cells/prominent ballooning.

Therefore, NASH refers to a NAFLD condition characterized by the following liver biopsy-derived grades: NAS≥3, with at least 1 point in steatosis, at least 1 point in lobular inflammation and at least 1 point in the hepatocyte ballooning scores.

More severe forms of NASH are also characterized by higher grades in one of the S, LI and HB scores described above, and/or the presence of liver fibrosis. In particular, “active NASH” refers to a NASH characterized by the following liver biopsy-derived grades: NAS≥4, with at least 1 point in steatosis, at least 1 point in lobular inflammation and at least 1 point in the hepatocyte ballooning scores.

“Liver fibrosis” refers to the presence of fibrous connective tissue at microscopic examination of a stained (H&E, trichrome or picrosirius red staining) slice of a liver biopsy. In the context of the present invention, the term “fibrosis stage” denotes the localization and extent of liver fibrosis at histological exam, as follows:
Perisinusoidal or periportal fibrosis 1
Mild perisinusoidal fibrosis (zone 3) 1a
Moderate perisinusoidal fibrosis (zone 3) 1b
Portal/periportal fibrosis 1c
Perisinusoidal and portal/periportal fibrosis 2
Bridging fibrosis 3
Cirrhosis 4

Alternatively, the fibrosis stage may be referred to as follows in the context of the present invention:
F=0: no fibrosis
F=1: minimal fibrosis
F=2: significant fibrosis
F=3: moderate fibrosis
F=4: severe fibrosis (i.e. cirrhosis)

In a particular embodiment, the present invention is used to diagnose NASH, classify a subject as a receiver or non-receiver of a treatment of NASH, or to monitor the efficiency of a treatment for NASH.

According to the present invention, the term NASH refers, without limitation, to different stages of NASH, including NASH, severe NASH, active NASH, fibrosing NASH and active NASH with significant fibrosis (i.e. an active NASH characterized by liver fibrosis stage of 2 or of more than 2, such as a fibrosis stage equal to 2, 3 or 4). The method of the present invention can be used in the context of all these kinds of NASH.

The method of the invention comprises:
i) measuring liver fibrosis of said subject with a physical method, and
ii) measuring the level of at least one circulating marker in a body fluid sample of said subject.

Step i) can be conducted thanks to a number of methods well known in the art. Illustrative methods include, without limitation, medical imaging and/or clinical measurement. In a particular embodiment, the physical method is elastometry. Elastometry method can further particularly be selected from the group consisting of Acoustic Radiation Force, Impulse imaging (ARFI imaging), transient elastography (TE) and MRI stiffness. In a particular embodiment of the invention, the physical method is transient elastography, which measures the difference in velocity of elastic shear wave propagation in the liver. According to a preferred method, transient elastography (such as FIBROSCAN®) is used, a technique used to assess liver hardness or stiffness, measured in kilopascal (kPa) and correlated to fibrosis, without invasive investigation. TE results (such as FIBROSCAN® results) can range from 2.5 kPa to 75 kPa. Between 90-95% of healthy subjects without liver disease will have a liver stiffness measurement<7.0 kPa. In a particular embodiment, liver stiffness is conducted as provided in the experimental part of the present application.

Step ii) comprises the measure of at least one circulating marker from a body fluid sample of the subject. The biological fluid can be a sample of blood, of a blood-derived fluid (for example serum or plasma, in particular platelet-free plasma, e.g. a cell-free, citrate-derived platelet-free plasma sample), of saliva, of cerebrospinal fluid or of urine. In a particular embodiment, the body fluid is blood, plasma or serum, deprived of platelets or not. One skilled in the art will know from which body fluid a specific circulating marker should be measured. For example for the level of specific markers mentioned below, hsa-miR34a, alpha 2 macroglobuline (A2M) and YKL-40 can be measured from serum and glycated haemoglobin (HbA1c) can be measured from blood.

In a particular embodiment, step ii) comprises measure of the level of at least one of the circulating markers disclosed in WO2017046181 and in WO2017167934.

In a particular embodiment, step i) comprises the measure of the level of at least one circulating marker selected in the group consisting of hsa-miR193 (such as hsa-miR193b-3p), hsa-miR34a , A2M, YKL-40 and HbA1c. In a variant of this embodiment, step i) comprises the measure of the level of hsa-miR34a or hsa-miR193, in particular of fsa-miR34a. In another particular embodiment, step i) comprises the measure of the level of at least two circulating marker selected in the group consisting of hsa-miR193 (such as hsa-miR193b-3p), hsa-miR34a, A2M, YKL-40 and HbA1c. In a variant of this embodiment, step i) comprises the measure of the level of hsa-miR34a and A2M. In yet another embodiment, step i) comprises the measure of the level of at least three circulating marker selected in the group consisting of hsa-miR193 (such as hsa-miR193b-3p), hsa-miR34a, A2M, YKL-40 and HbA1c. In a variant of this embodiment, step i) comprises the measure of the level of hsa-miR34a, A2M and YKL-40.

It should be understood that in all embodiments and variants disclosed herein, hsa-miR34a can more particularly be hsa-miR34a-5p.

In another particular embodiment, step i) comprises the measure of the level of hsa-miR34a, A2M, YKL-40 and HbA1c. In the present application, the combination of these four markers is also referred to as NIS4. It is herein shown that combining these measures with the measure of liver stiffness of step i) leads to an unexpected improvement of the specificity of the diagnosis of NASH, of the classification of a subject as a receiver or non-receiver of a treatment of NASH, or of the monitoring of the efficiency of a treatment of NASH.

In a particular embodiment, the measures of the levels of the circulating markers are used in a logistic function to calculate a score, for example as provided in the application WO2017167934. This score and the measure of liver stiffness can be combined in a logistic function to determine a further improved score.

In a particular embodiment, a NIS4 score is used in combination to the measured stiffness. The NIS4 score is more particularly calculated as provided in WO2017167934, as follows:

S1˜e^{Y1}/1+e^{Y1}

wherein:

### Y1=k+a*A+b*B+c*C+d*D

wherein:
S1 is the NASH score 1 or the NIS4 score;
A is the serum level of hsa-miR-34a-5p in Cq;
B is the serum level of alpha 2 macroglobulin in g/L;
C is the serum level of YKL-40 in ng/mL,
D is the level of HbA1c in percent (e.g. D is equal to 10 if measured HbA1c percentage is 10%);
k is the constant of the logistic function
a is a coefficient associated to the serum level of hsa-miR-34a-5p;
b is a coefficient associated to the serum level of alpha 2 macroglobulin;
c is a coefficient associated to the serum level of YKL-40;
d is a coefficient associated to the level of HbA1c.

The improved score of the present invention may be calculated as follows, based on a NIS4 score and the measure of liver stiffness, thanks to the following logistic function:

$$S 2 ∼ \frac{{e}^{Y 2}}{1 + {e}^{Y 2}}$$

### Y2=1+e*S1+PFS

wherein:
S1 is the NIS4 score;
FS is the measured stiffness in kPa;
1 is the constant of the logistic function;
e is a coefficient associated to the NIS4 score; and
f is a coefficient associated to the measured liver stiffness.

If S2 is greater or equal to a threshold value, the subject is classified as having or potentially having a NASH and/or is classified as a receiver of a treatment for NASH. If S2 is lower than a threshold value, the subject may be classified as a receiver or non-receiver, in particular as a non-receiver, of a treatment for NASH and/or the subject is classified as a receiver, or potential receiver, of diet and lifestyle advices for managing his/her NASH.

In a particular embodiment, 1 has a value from −3.6296 to −0.2985, e has a value from 1.539 to 5.629, and f has a value from 0.0107 to 0.3229.

It is further herein shown that the removal of one marker among the markers of NIS4, namely the removal of Hb1Ac, has no major impact on the predictive value of the method of the invention, in particular no deep impact on the sensitivity and on the specificity of the method. A further advantage of this removal of HblAc from the markers measured is that it allows the possibility of conducting a measure from only one body fluid sample, namely serum, instead of two different body fluid samples (since Hb1Ac levels are determined from blood). All these elements advantageously provide a method easier to implement, more cost-effective, and less prone to test-to-test variations. Therefore, in a particular embodiment, the method of the invention comprises the measure of liver stiffness and the measure of the level of miR34a, A2M and YKL40. Collectively, miR34a, A2M and YKL40 markers are referred as NIS3.

In a particular embodiment, an improved score may be calculated as follows, based on NIS3 measures and the measure of liver stiffness, thanks to the following logistic function:

$$S 3 ∼ \frac{{e}^{Y 3}}{1 + {e}^{Y 3}}$$

Y3=m+g*A+h*B+i*C+j*FS

wherein:
A is the serum level of hsa-miR34a in Cq;
B is the serum level of alpha 2 macroglobulin in g/L;
C is the serum level of YKL-40 in ng/mL;
FS is the measured stiffness in kPa;
m is the constant of the logistic function;
g is a coefficient associated to the serum level of hsa-miR-34a-5p;
h is a coefficient associated to the serum level of alpha 2 macroglobulin;
i is a coefficient associated to the serum level of YKL-40; and
j is a coefficient associated to the measured stiffness.

If S3 is greater or equal to a threshold value, the subject is classified as having or potentially having a NASH and/or is classified as a receiver of a treatment for NASH. If S3 is lower than a threshold value, the subject may be classified as a receiver or non-receiver, in particular as a non-receiver, of a treatment for NASH and/or the subject is classified as a receiver, or potential receiver, of diet and lifestyle advices for managing his/her NASH.

In a particular embodiment, m has a value from −2.52 to 35.38, g has a value from −1.2061 to −0.0355, h has a value from 0.3104 to 1.7716, i has a value from −0.0015 to 0.0207, and j has a value from −0.0096 to 0.3465.

Moreover, and highly unexpectedly, it is further herein shown that the removal of yet another one marker among the markers of NIS3, namely the removal of YKL, has no major impact on the predictive value of the method of the invention, while it provides a better specificity over the method based on NIS3 measures. This advantageously provides a method even easier to implement (only two markers to measure), more cost-effective, and less prone to test-to-test variations. Therefore, in a particular embodiment, the method of the invention comprises the measure of liver stiffness and the measure of the level of miR34a and A2M. Collectively, miR34a and A2M markers are referred as NIS2.

In a particular embodiment, an improved score may be calculated as follows, based on NIS2 measures and the measure of liver stiffness, thanks to the following logistic function:

$$S 4 ∼ \frac{{e}^{Y 4}}{1 + {e}^{Y 4}}$$

### Y4=n+o*A+p*B+q*FS

wherein:
A is the serum level of hsa-miR-34a-5p in Cq;
B is the serum level of alpha 2 macroglobulin in g/L;
FS is the measured stiffness;
n is the constant of the logistic function;
o is a coefficient associated to the serum level of hsa-miR-34a-5p;
is a coefficient associated to the serum level of alpha 2 macroglobulin;
q is a coefficient associated to the measured stiffness.

If S4 is greater or equal to a threshold value, the subject is classified as having or potentially having a NASH and/or is classified as a receiver of a treatment for NASH. If S4 is lower than a threshold value, the subject may be classified as a receiver or non-receiver, in particular as a non-receiver, of a treatment for NASH and/or the subject is classified as a receiver, or potential receiver, of diet and lifestyle advices for managing his/her NASH.

In a particular embodiment, n has a value from −9.607 to 35.175, o has a value from −1.2012 to 0.2127, p has a value from 0.4424 and 1.9381 and q has a value from 0.0258 and 0.3617.

In some embodiments, thanks to the methods of the invention, a decision may be taken to give life style recommendations to a subject (such as a food regimen or providing physical activity recommendations), to medically take care of a subject (e.g. by setting regular visits to a physician or regular examinations, for example for regularly monitoring markers of liver damage), or to administer at least one NASH or liver fibrosis therapy to a subject. In a particular embodiment, a decision may be taken to give life style recommendations to a subject or to administer at least one NASH or liver fibrosis therapy.

The invention thus further relates to an anti-NASH or anti-fibrotic compound for use in a method for treating NASH, NASH with fibrosis, or active NASH with significant fibrosis in a subject in need thereof, wherein the subject has been identified thanks to a method according to the invention.

In particular, the invention relates to an anti-NASH compound for use in a method for treating NASH, NASH with fibrosis, or active NASH with significant fibrosis in a subject in need thereof, wherein the subject has been classified as a receiver of said treatment thanks to a method according to the invention.

Illustrative anti-NASH and anti-fibrotic compounds are listed below:
a compound of formula (I):

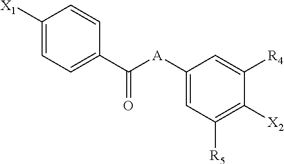

wherein:
X1 represents a halogen, a R1, or G1-R1 group;
A represents a CH=CH or a CH2-CH2 group;
X2 represents a G2-R2 group;
G1 and G2, identical or different, represent an atom of oxygen or sulfur;
R1 represents a hydrogen atom, an unsubstituted alkyl group, an aryl group or an alkyl group that is substituted by one or more halogen atoms, an alkoxy or an alkylthio group, cycloalkyl groups, cycloalkylthio groups or heterocyclic groups;
R2 represents an alkyl group substituted by at least a -COOR3 group, wherein R3 represents a hydrogen atom, or an alkyl group that is substituted or not by one or more halogen atoms, cycloalkyl groups, or heterocyclic groups.
R4 and R5, identical or different, representing an alkyl group that is substituted or not by one or more halogen atoms, cycloalkyl groups, heterocyclic groups;
or a pharmaceutically acceptable salt thereof;
Acetyl-CoA carboxylase inhibitors like GS-0976, ND-654, AC-8632, PF05175157, CP640186, gemcabene, MK-4074, and PF05175157.
Adenosine A3 receptor agonists like 2-(1-Hexynyl)-N-methyladenosine, Piclidenoson CF101 (IB-MECA), Namodenoson CF-102, 2-Cl-IB-MECA, CP-532,903, Inosine, LUF-6000, and MRS-3558.
Aldosterone antagonists and mineralocorticoid receptor antagonists like Apararenone (MT 3995), Amiloride, Spironolactone, Eplerenone, Canrenone and potassium canrenoate, progesterone, drospirenone, gestodene, and benidipine.
AMP activated protein kinase stimulators like PXL-770, MB-11055 Debio-0930B metformin, CNX-012, O-304, mangiferin calcium salt, eltrombopag, carotuximab, and Imeglimin.
Amylin receptor agonist and Calcitonin receptor agonists include, but are not limited to, KBP-042 and KBP-089.
Antisense oligonucleotide targeting transforming growth factor beta 2 include, but are not limited to ASPH-0047, IMC-TR1 and ISTH-0047.
Angiopoietin-related protein-3 inhibitors like ARO-ANG3, IONIS-ANGGPTL3-LRx or AKCEA-ANGPTL3LRx, evinacumab, and ALN-ANG.
Anti-LPS antibodies like IMM-124-E
Apical sodium-codependent bile acid transporter inhibitors like A-4250, volixibat, maralixibat formely SHP-625, GSK-2330672, elobixibat, and CJ-14199.
Betaine anhydrous or RM-003;
Bile acids like obeticholic acid (OCA) and UDCA, norursodeoxycholic acid, and ursodiol.
Bioactive lipids like 5-hydroxyeicosapentaenoic acid (15-HEPE, DS-102), unsaturated fatty acids such as 25 arachidonic acid, icosapentethyl ester, eicosapentaneoic acid, and docosahexaenoic acid.
Cannabinoid CB1 receptor antagonists like GRC-10801, MRI-1569, MRI-1867, DBPR-211, AM-6527 : AM-6545, NESS-11-SM, CXB-029, GCC-2680, TM-38837, Org-50189, PF-514273, BMS-812204, ZYO-1, AZD-2207, AZD-1175, otenabant, ibipinabant, surinabant, rimonabant, drinabant, SLV-326, V-24343, and O-2093.
Cannabinoid CB2 receptor mimetics like anabasum (Resunab, JKT-101).
Dual cannabinoid CB1 receptor/iNOS inhibitor
Caspase inhibitors like emricasan, belnacasan, nivocasan, IDN-7314, F-573, VX-166, YJP-60107, MX-1122, IDN-6734, TLC-144, SB-234470, IDN-1965, VX-799, SDZ-220-976, and L-709049.
Cathepsin inhibitors like VBY-376, VBY-825, VBY-036, VBY-129, VBY-285, Org-219517, LY3000328, RG-7236, and BF/PC-18.
CCR antagonists like cenicriviroc (CCR2/5 antagonist), PG-092, RAP-310, NCB-10820, RAP-103, PF-04634817, and CCX-872.
CCR3 chemokine modulators and eotaxin 2 ligand inhibitors.
Diacylglycerol-0-acyltransferase (DGAT) inhibitors like IONIS-DGAT2Rx formely ISIS-DGAT2Rx, LY-3202328, BH-03004, KR-69530, OT-13540, AZD-7687, ABT-046.
Dipeptidyl peptidase IV (DPP4) inhibitors like evogliptin, vidagliptin, fotagliptin, alogliptin, saxagliptin, tilogliptin, anagliptin, sitagliptin, retagliptin, melogliptin, gosogliptin, trelagliptin, teneligliptin, dutogliptin, linagliptin, gemigliptin, yogliptin, betagliptin, imigliptin, omarigliptin, vidagliptin, and denagliptin.
Insulin ligand and insulin receptor agonists.
Insulin sensitizer and MCH receptor-1 antagonis
Dual NOX (NADPH oxidase) 1&4 inhibitors like GKT-831 (2-(2-chlorophenyl)-4-[3-(dimethylamino)phenyl]-5-methyl-1H-pyrazolo [4,3-c]pyridine-3,6(2H,5H)-dione), formerly GKT137831, and GKT-901.
Extracellular matrix protein modulators likeCNX-024, CNX-025, and SB-030.
Stearoyl CoA desaturase-1 inhibitors/fatty acid bile acid conjugates (FABAC);
Farnesoid X receptor (FXR) agonists like obeticholic acid (OCA), GS-9674, LJN-452, EDP-305, AKN-083, INT-767, GNF-5120, LY2562175, INV-33, NTX-023-1, EP-024297, Px-103, and SR-45023.
Fatty acids like omega-3 fatty acids, Omacor or MF4637, fish oils, poly unsatured fatty acids (efamax, optiEPA).
Fatty Acid Synthase (FAS) inhibitors like TVB-2640; TVB-3199, TVB-3693BZL-101, 2-octadecynoic acid, MDX-2, Fasnall, MT-061, G28UCM, MG-28, HS-160, GSK-2194069, KD-023, and cilostazol.

In a particular embodiment, the FAS inhibitor is a compound selected in the following list of compounds:

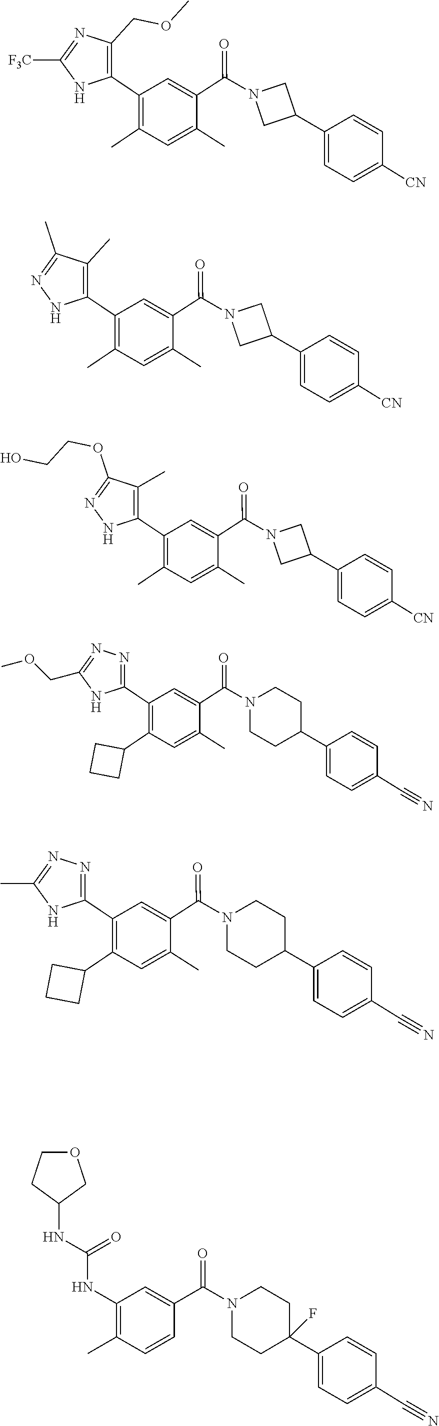 [SMILES: COCc1nc(C(F)(F)F)[nH]c1-c1cc(C(=O)N2CC(c3ccc(C#N)cc3)C2)c(C)cc1C ; Cc1cc(C)c(-c2[nH]nc(C)c2C)cc1C(=O)N1CC(c2ccc(C#N)cc2)C1 ; Cc1cc(C)c(-c2[nH]nc(OCCO)c2C)cc1C(=O)N1CC(c2ccc(C#N)cc2)C1 ; COCc1nnc(-c2cc(C(=O)N3CCC(c4ccc(C#N)cc4)CC3)c(C)cc2C2CCC2)[nH]1 ; Cc1nnc(-c2cc(C(=O)N3CCC(c4ccc(C#N)cc4)CC3)c(C)cc2C2CCC2)[nH]1 ; Cc1ccc(C(=O)N2CCC(F)(c3ccc(C#N)cc3)CC2)cc1NC(=O)NC1CCOC1]

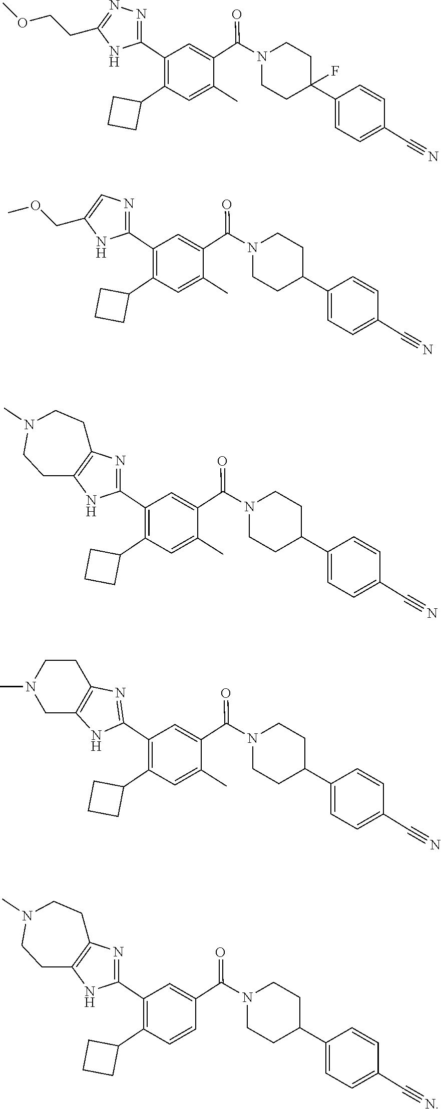 [SMILES: COCCc1nnc(-c2cc(C(=O)N3CCC(F)(c4ccc(C#N)cc4)CC3)c(C)cc2C2CCC2)[nH]1 ; COCc1cnc(-c2cc(C(=O)N3CCC(c4ccc(C#N)cc4)CC3)c(C)cc2C2CCC2)[nH]1 ; Cc1cc(C2CCC2)c(-c2nc3c([nH]2)CCN(C)CC3)cc1C(=O)N1CCC(c2ccc(C#N)cc2)CC1 ; Cc1cc(C2CCC2)c(-c2nc3c([nH]2)CN(C)CC3)cc1C(=O)N1CCC(c2ccc(C#N)cc2)CC1 ; CN1CCc2nc(-c3cc(C(=O)N4CCC(c5ccc(C#N)cc5)CC4)ccc3C3CCC3)[nH]c2CC1]

### and TVB-2640.

In another particular embodiment, the FAS inhibitor is selected from:

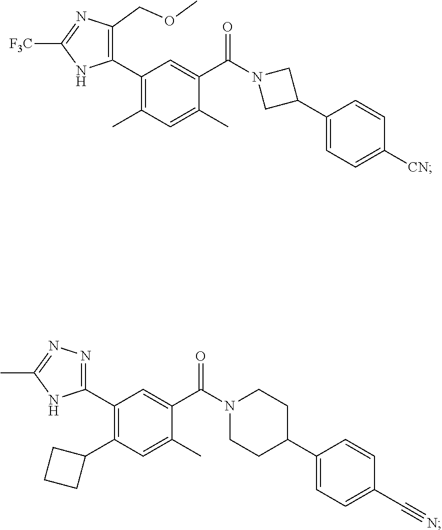 [SMILES: COCc1nc(C(F)(F)F)[nH]c1-c1cc(C(=O)N2CC(c3ccc(C#N)cc3)C2)c(C)cc1C ; Cc1nnc(-c2cc(C(=O)N3CCC(c4ccc(C#N)cc4)CC3)c(C)cc2C2CCC2)[nH]1]

### and TVB-2640.

In a particular embodiment, the FAS inhibitor is TVB-2640.
Fibroblast Growth Factor 19 (FGF-19) receptor ligand or functional engineered variant of FGF-19
Fibroblast Growth Factor 19 (FGF-19) recombinants like NGM-282
Fibroblast Growth Factor 21 (FGF-21) agonists like PEG-FGF21 formely BMS-986036, YH-25348, BMS-986171, YH-25723, LY-3025876, and NNC-0194-0499.
Galectin 3 inhibitors like GR-MD-02, TD-139, ANG-4021, Galectin-3C, LJPC-201, TFD-100, GR-MD-03, GR-MD-04, GM-MD-01, GM-CT-01, GM-CT-02, Gal-100, and Gal-200.
Glucagon-like peptide-1 (GLP-1) analogs like semaglutide, liraglutide, exenatide, albiglutide, dulaglutide, lixisenatide, loxenatide, efpeglenatide, taspoglutide, MKC-253, DLP-205, ORMD-0901.
Glucagon-like peptide-1 (GLP-1) receptor agonists like LY-3305677, and Oxyntomodulin long acting.
G-protein coupled receptor (GPCR) modulators; CNX-023.
G-protein coupled receptor 84 antagonist (GPR84 antagonist), connective tissue growth factor ligand inhibitor and Free fatty acid receptor 1 agonist (FFAR1 agonist) like PBI-4050, PBI-4265, PBI-4283, and PBI-4299.
Growth hormone
Hedgehog cell-signalling pathway inhibitors like Vismodegib, TAK-441, IPI-926, Saridegib, Sonidegib/Erismodegib, BMS-833923/XL139, PF-04449913, Taladegib/LY2940680, ETS-2400, SHR-1539, and CUR61414.
Ileal sodium bile acid cotransporter inhibitors like A-4250, GSK-2330672, volixibat, CJ-15 14199, and elobixibat
Immunomodulators like PBI-4050, PBI-4265, PBI-4283, PBI-4299 and AIC-649.
Insulin sensitizer and MCH receptor-1 antagonist like MSDC-0602k, MSDC-0602, CSTI-100 and AMRI
Integrin inhibitors; integrin inhibitors of Pliant Therapeutic, integrin inhibitors of Indalo Therapeutics, integrin inhibitors of St Louis University, ProAgio, and GSK-3008348.
Ketohexokinase inhibitors like JNJ-28165722, JNJ-42065426; JNJ-42152981, JNJ-42740815, JNJ-42740828, and PF-06835919.
Leukotriene (LT)/Phosphodiesterase (PDE)/Lipoxygenase (LO) inhibitors like tipelukast (formely MN-001), tomelukast, sulukast, masilukast, zafirlukast, pranlukast, montelukast, gemilukast, verlukast, aklukast, pobilikast, cinalukast, and iralukast.
Lysyl oxidase homolog 2 inhibitors like Rappaport, InterMune, Pharmaxis, AB-0023,

Simtuzumab, PXS-5382A, and PXS-5338.
Macrolides: solithromycin, azithromycin, and erythromycin.
Macrophage mannose receptor modulators like AB-0023, MT-1001, [18F]FB18mHSA, Xemys, technetium Tc 99m tilmanocept, and CDX-1307.
Methyl CpG binding protein 2 modulator and transglutaminase inhibitors include, but are not limited to, cysteamine, EC Cysteamine, enteric-coated cysteamine bitartrate, cysteamine bitartrate (enteric-coated), Bennu, cysteamine bitartrate (enteric-coated), Raptor, cysteamine bitartrate, DR Cysteamine, delayed release enteric coated cysteamine bitartrate, mercaptamine, mercaptamine (enteric-coated), Bennu, mercaptamine (enteric-coated), Raptor, RP-103, RP-104, PROCYSBI, and mercaptamine (enteric-coated).
miRNA antagonists like RG-125 formely AZD4076, RGLS-5040, RG-101, MGN-5804, and MRG-201.
Metalloproteinase 9 (MMP9) stimulator like MMP9 stimulator of Elastomic Ab.
Mitochondrial carrier family inhibitor and Mitochondrial phosphate carrier protein inhibitor include, but are not limited to TRO-19622, Trophos, olesoxime, RG-6083, or RO-7090919.
Myeloperoxidase inhibitors include, but are not limited to PF-06667272
Monoclonal antibodies: bertilimumab, NGM-313, IL-20 targeting mAbs, fresolimumab (antiTGFβ) formely GC1008, timolumab formely BTT-1023, namacizumab, omalizumab, ranibizumab, bevacizumab, lebrikizumab, epratuzumab, felvizumab, matuzumab, monalizumab, reslizumab, and inebilizumab.
Monoclonal antibodies like anti-IL20 mAbs, anti-TGFβ antibodies, anti-CD3 antibodies, anti-LOXL2 antibodies and anti-TNF antibodies.
mTOR modulators like MSDC-0602, AAV gene therapy co-administered with SVP-sirolimus.
NAD-dependent deacetylase sirtuin stimulator, PDE 5 inhibitor like NS-0200.
NF-kappa B inhibitors like LC-280126.
Nicotinic acid like Niacin or Vitamine B3
Nicotinic Acid Receptor (GPR109) Agonists like ARI-3037M0, MMF, LUF 6283, Acifran, IBC 293, MK-1903, GSK256073, MK-6892, MK-0354, SLx-4090, lomitapide, lexibulin, apabetalone, acifran, laropiprant, daporinad, anacetrapib, NCB-19602, ST-07-02, lomefloxacin, Niacin, and controlled release/laropiprant,
nitazoxanide (NTZ), its active metabolite tizoxanide (TZ) or other prodrugs of TZ such as RM-5061,
non-steroid anti-inflammatory drugs (NSAIDs) include, but are not limited to F-351, salicylates (aspirin), acetaminophen, propionic acid derivatives (ibuprofen, naproxen), acetic acid derivatives (indomethacin, diclofenac), enolic acid derivatives (piroxicam, phenylbutazone), anthranilic acid derivatives (meclofenalmic acid, flufenamic acid), selective 25 COX-2 inhibitors (celecoxib, parecoxib), and sulfonanilides (nimesulide).
nuclear receptor ligands like DUR-928 formely DV 928.
P2Y13 protein agonists like CER-209
PDGFR modulators like BOT-501 and BOT-191.
Phenylalanine hydroxylase stimulators like Pegvaliase, sapropterin, AAV-PAH, CDX-6114, sepiapterin, RMN-168, ALTU-236, ETX-101, HepaStem, rolipram, and alprostadil
Protease-activated receptor (PAR)-2 antagonists; PZ-235, and NP-003.
Protein kinase modulators like CNX-014, MB-11055, ALF-1, mangiferin, amlexanox, GS-444217, REG-101, and valine.
PPAR alpha agonists like fenofibrate, ciprofibrate, pemafibrate, gemfibrozil, clofibrate, binifibrate, clinofibrate, clofibric acid, nicofibrate, pirifibrate, plafibride, ronifibrate, theofibrate, tocofibrate, and SR10171;
PPAR gamma agonists like Piogli axone, deuterated pioglitazone, Rosiglitazone, efatutazone, ATx08-001, OMS-405, CHS-131, THR-0921, SER-150-DN, KDT-501, GED-0507-34-Levo, CLC-3001, and ALL-4.
PPAR delta agonists like GW501516 (Endurabol or ({4-[({4-methyl-2-[4-(trifluoromethyl)phenyl]-1,3-thiazol-5-yl}methyl)sulfanyl]-2-methylphenoxy}acetic acid)) or MBX8025 (Seladelpar or {2-methyl-4-[5-methyl-2-(4-trifluoromethyl-phenyl)-2H-[1,2,3]triazol-4-ylmethylsylfanyl]-phenoxy}-acetic acid) or GW0742 ([4-[[[2-[3-fluoro-4-(trifluoromethyl)phenyl]-4-methyl-5-thiazolyl]methyl]thio]-2-methyl phenoxy]acetic acid) or L165041 or HPP-593 or NCP-1046.
PPAR alpha/gamma agonists (also named glitazars), like Saroglitazar, Aleglitazar, Muraglitazar, Tesaglitazar, and DSP-8658.
PPAR alpha/delta agonists like Elafibranor, and T913659.
PPAR gamma/delta like conjugated linoleic acid (CLA), T3D-959.
PPAR alpha/gamma/delta agonists or PPARpan agonists: IVA337 or TTA (tetradecylthioacetic acid) or Bavachinin or GW4148 or GW9135, or Bezafibrate or Lobeglitazone, or CS038.
Prebiotic fibers, probiotics
Pregnane X receptors like Rifampicin.
Rho-associated protein kinase 2 (ROCK2) inhibitors: KD-025, TRX-101, BA-1049, LYC-53976, INS-117548, and RKI-1447.
signal-regulating kinase 1 (ASK1) inhibitors; GS-4997
Sodium-glucose transport (SGLT) 2 inhibitors: remogliflozin, dapagliflozin, empagliflozin, ertugliflozin, sotagliflozin, ipragliflozin, tianagliflozin, canagliflozin, tofogliflozin, janagliflozin, bexagliflozin, luseogliflozin, sergliflozin, HEC-44616, AST-1935, and PLD-101.
stearoyl CoA desaturase-1 inhibitors/fatty acid bile acid conjugates: aramchol, GRC-9332, steamchol, TSN-2998, GSK-1940029, and XEN-801.
thyroid receptor β (THR β) agonists: VK-2809, MGL-3196, MGL-3745, SKL-14763, sobetirome, BCT-304, ZYT-1, MB-07811, and eprotirome.
Toll Like Receptor 4 (TLR-4) antagonists like naltrexone, JKB-121, M-62812, resatorvid, dendrophilin, CS-4771, AyuV-1, AyuV-25, NI-0101, EDA-HPVE7, and eritoran.
Tyrosine kinase receptor (RTK) modulators; CNX-025 ; KBP-7018
Urate anion exchanger 1 inhibitors and xanthine oxidase inhibitors like lesinurad, RLBN-1001, verinurad, KUX-1151, and lesinurad+allopurinol.
Vascular adhesion protein-1 (VAP-1) inhibitors like PXS-4728A, CP-664511, PRX-167700, ASP-8232, RTU-1096, RTU-007, and BTT-1023.
Vitamin D receptor (VDR) agonists like calciferol, alfacalcidol, 1,25-dihydroxyvitamin D3, Vitamin D2, Vitamin D3, calcitriol, Vitamin D4, Vitamin D5, dihydrotachysterol, calcipotriol; tacalcitol 1,24-dihydroxyvitamin D3, and paricalcitol.
Vitamin E and isoforms, vitamin E combined with vitamin C and atorvastatin.

Other anti-NASH agents include KB-GE-001 and NGM-386 and NGM-395 and NC-10 and TCM-606F. Further anti-NASH agents include icosabutate, NC-101, NAIA-101 colesevelam, and PRC-4016. Other anti-fibrotic agents include HEC-585, INV-240, RNAi therapeutic (Silence Therapeutics) and SAMiRNA program (Bioneer Corp). Other illustrative antifibrotic agents include pirfenidone or receptor tyrosine kinase inhibitors (RTKIs) such as Nintedanib, Sorafenib and other RTKIs, or angiotensin II (AT1) receptor blockers, or CTGF inhibitor, or any antifibrotic compound susceptible to interfere with the TGFβ and BMP-activated pathways including activators of the latent TGFβ complex such as MMP2, MMP9, THBS1 or cell-surface integrins, TGFβ receptors type I (TGFBRI) or type II

(TGFBRII) and their ligands such as TGFf3, Activin, inhibin, Nodal, anti-Müllerian hormone, GDFs or BMPs, auxiliary co-receptors (also known as type III receptors), or components of the SMAD-dependent canonical pathway including regulatory or inhibitory SMAD proteins, or members of the SMAD-independent or non-canonical pathways including various branches of MAPK signaling, TAK1, Rho-like GTPase signaling pathways, phosphatidylinositol-3 kinase/AKT pathways, TGFβ-induced EMT process, or canonical and non-canonical Hedgehog signaling pathways including Hh ligands or target genes, or any members of the WNT, or Notch pathways which are susceptible to influence TGFβ.

In a particular embodiment the treatment of NASH, NASH with fibrosis, or active NASH with significant fibrosis or liver fibrosis comprises administering a compound of formula (I) selected in the group consisting of 1-[4-methylthiophenyl]-3-[3,5-dimethyl-4-carboxydimethylmethyloxyphenyl]prop-2-en-1-one, 1-[4-methylthiophenyl]-3-[3,5-dimethyl-4-isopropyloxycarbonyldimethylmethyloxyphenyl]prop-2-en-1-one, 1-[4-methylthiophenyl]-3-[3,5-dimethyl-4-tertbutyloxycarbonyldimethylmethyloxyphenyl]prop-2-en-1-one, 1-[4-trifluoromethylphenyl]-3-[3,5 -dimethyl-4-tertbutyloxycarbonyl dimethylmethyloxyphenyl]prop-2-en-1-one, 1-[4-trifluoromethylphenyl]-3-[3,5-dimethyl-4-carboxydimethylmethyloxyphenyl]prop-2-en-1-one, 1-[4-trifluoromethyl oxyphenyl]-3-[3,5-dimethyl-4-tertbutyloxycarbonyldimethylmethyloxy phenyl]prop-2-en-1-one, 1-[4-trifluoromethyloxyphenyl]-3-[3,5 -dimethyl-4-carboxydimethylmethyloxyphenyl]prop-2-en-1-one, 2-[2,6-dimethyl-4-[3-[4-(methylthio)phenyl]-3-oxo-propyl]phenoxy]-2-methylpropanoic acid, and 2-[2,6-dimethyl-4-[3-[4-(methylthio) phenyl]-3-oxo-propyl]phenoxy]-2-methyl-propanoic acid isopropyl ester; or a pharmaceutically acceptable salt thereof. In a further particular embodiment of the invention, the compound of formula (I) is 1-[4-methylthiophenyl]-3-[3,5-dimethyl-4-carboxydimethylmethyloxy phenyl]prop-2-en-1-one or a pharmaceutically acceptable salt thereof.

In particular, the invention relates to a combination product comprising at least an anti-NASH and/or an anti-fibrotic agent for use in a method for treating NASH, NASH with fibrosis, or active NASH with significant fibrosis in a subject in need thereof, wherein the subject has been classified as a receiver of said treatment thanks to a method according to the invention.

In a more particular embodiment, the invention relates to the treatment of NASH, NASH with fibrosis, or active NASH with significant fibrosis with a combination product comprising at least one agent selected from the group of anti-NASH and/or anti-fibrotic compounds, or pharmaceutically acceptable salts thereof.

In a more particular embodiment, the invention relates to the treatment of NASH, NASH with fibrosis, or active NASH with significant fibrosis with Elafibranor.

In a further embodiment the treatment of NASH, NASH with fibrosis, or active NASH with significant fibrosis comprises administering NTZ, TZ, vitamin E or pioglitazone, obeticholic acid, elafibranor, selonsertib, saroglitazar and/or cenicrivoc.

In a further embodiment, the treatment of NASH, NASH with fibrosis, or active NASH with significant fibrosis comprises administering NTZ or TZ, in particular NTZ.

In a further particular embodiment, a combination treatment is conducted. In another particular embodiment, the treatment of NASH, NASH with fibrosis, or active NASH with significant fibrosis comprises administering Elafibranor combined with one or more other NASH or anti-liver fibrosis compound. In yet another embodiment, the treatment of NASH, NASH with fibrosis, or active NASH with significant fibrosis comprises administering Elafibranor combined with at least one compound selected in the group consisting of NTZ, TZ, vitamin E or pioglitazone, obeticholic acid, selonsertib, saroglitazar, bezafibrate and cenicrivoc. In yet another embodiment, the treatment of NASH, NASH with fibrosis, or active NASH with significant fibrosis comprises administering Elafibranor combined with NTZ.

### EXAMPLES

### Materials And Methods

### A. Clinical Samples

The inventors had access to human blood samples from subjects with a liver biopsy and associated clinical and biological data from the RESOLVE-IT study. RESOLVE-IT is a Multicenter, Randomized, Double-Blind, Placebo-Controlled Phase III Study (NCT02704403) to Evaluate the Efficacy and Safety of Elafibranor in Patients with Nonalcoholic Steatohepatitis (NASH) and fibrosis. The final objective of the randomized placebo controlled RESOLVE-IT trial (NCT02704403) is to assess efficacy and safety of Elafibranor in about 2000 patients with active NASH (NAS≥4) and significant fibrosis (F-stage≥2) determined by centralized scoring of a liver biopsy collected during the screening period or less than 6 months before the inclusion visit. Compensated (F-stage=4) and decompensated cirrhotic patients were not included in the active phase of the trial. The study was approved by appropriate regulatory bodies all patients had given informed consent for participation. An inclusion liver biopsy was used for examination and scoring of histological lesions. Patients characteristics and distribution across histological spectrum of NAFLD are presented in Table 1.

321 patients were included in the biostatistical analysis: 21.5% NTBT and 78.5% TBT, with F1 (n=69), F2 (n=123) and F3 (n=129) according to biopsy data.

Patient characteristics of the RESOLVE-IT cohort used in the present study are outlined in Table 1.

| | | CHI3L1/ | hsa-miR-34a- | | |
|---|---|---|---|---|---|
| A2M | HbA1c | YKL-40 | 5p | NIS4_score | Fibroscan |
| g/L | % | ng/mL | Cq | (0-1) | kPa |
| Min.: 0.740 | Min.: 4.600 | Min.: 3.1 | Min.: 28.43 | Min.: 0.05301 | Min.: 0.00 |
| 1st Qu.: 1.810 | 1st Qu.: 5.500 | 1st Qu.: 39.7 | 1st Qu.: 30.71 | 1st Qu.: 0.44215 | 1st Qu.: 7.00 |
| Median: 2.380 | Median: 6.100 | Median: 67.5 | Median: 31.40 | Median: 0.63874 | Median: 9.70 |
| Mean: 2.466 | Mean: 6.298 | Mean: 106.6 | Mean: 31.32 | Mean: 0.62953 | Mean: 12.04 |
| 3rd Qu.: 3.020 | 3rd Qu.: 6.800 | 3rd Qu.: 122.7 | 3rd Qu.: 31.95 | 3rd Qu.: 0.86221 | 3rd Qu.: 14.00 |
| Max.: 5.860 | Max.: 9.000 | Max.: 1600.0 | Max.: 33.58 | Max.: 1.00000 | Max.: 72.00 |

Patient characteristics, categorical variables are expressed in %, quantitative variables are expressed as absolute mean, minimal and maximal value, median, first and third quartile (Qu).

NASH and fibrosis were evaluated by a centrally-read liver biopsy taken within 6 months prior to randomization (if no historical biopsy is available for NASH diagnosis, a liver biopsy was performed during the Screening Period) with:
At least a score of 1 in each component of the NAS score (steatosis scored 0-3, ballooning degeneration scored 0-2, and lobular inflammation scored 0-3).
NAS≥4.
fibrosis stage of 1 or greater and below 4, according to the NASH CRN fibrosis staging system.

For patients with fibrosis stage 1, only patients at high risk of progression were included, meaning with a NAS score≥5 and 2 of the following conditions: persistent elevated alanine aminotransferase (ALT), obesity defined by a body mass index (BMI) z 30, metabolic syndrome (NCEP ATP III definition), type 2 diabetes, or homeostasis model assessment of insulin resistance (HOMA-IR)>6.

### B. Liver Biopsy and Histological Scoring

For RESOLVE-IT cohort, liver biopsies were collected at investigational centers of RESOLVE-IT trials. Histologic scoring according to NASH-CRN systems was centralized and performed by a trained pathologist at Hopital Beaujon (Paris France).

### C. Blood Sampling and Laboratory Testing

Blood samples used in this study were drawn from patients before treatment period. Blood collected in serum separating tube (SST) 8.5mL was processed one hour after 15 sampling by separating cell-free serum from blood cells by centrifugation between 1,300×g and 2,000×g for 10 minutes. The serum was then transferred to a new tube. Tubes were kept at −70° C. for the determination of YKL-40 and hsa-miR-34a-5p levels or at room temperature for the determination of alpha2-macroglobulin A2M.

Blood collected in EDTA collection tube was kept at room temperature before HbA1c determination.

### Fibroscan

Liver stiffness was evaluated with FIBROSCAN® (EchoSens, Paris), also called transient elastography, a technique used to assess liver hardness or stiffness (measured in kilopascal-kPa-correlated to fibrosis) without invasive investigation. FIBROSCAN® results range from 2.5 kPa to 75 kPa. Between 90-95% of healthy people without liver disease will have a liver scarring measurement <7.0 kPa.

Transient elastography was performed according to the manufacturer's recommendations: User manual for probe M+ E117M011.2—Version 2—April/2017, User manual for probe XL+ E117M013.3—Version 3—March/2018, User manual for the FIBROSCAN® 530 COMPACT E320M001.8—Version8—December/2017 (software version G 3.2). M or XL probe was used depending on patient's age, thoracic perimeter (TP) and skin capsula distance (SCD):

age<18, TP>75 cm: M probe,
age≥18, SCD<2.5 cm: M probe,
age≥18, 3.5 cm>SCD>2.5 cm: XL probe.

### D. Biochemical Analyses

YKL40 (also referred to as CHI3L1) was quantitatively determined in serum by an ELISA (Human Chitinase 3-like 1 Immunoassay Quantikine® ELISA Catalog Number DC3L10). Values were expressed as ng/mL.

Alpha2 macroglobulin levels were determined by nephelometry on a BN II system (Siemens Healthcare). Values were expressed as g/L.

HbA1c was determined by ion-exchange high performance liquid chromatography (HPLC) method (Menarini HA-8160 HbA1c auto-analyzer) and reported as a percentage of total haemoglobin.

### E. Quantitative RTqPCR of miR-34a-5p in Serum

RNA extraction was performed without additional centrifugation of serum samples.

RNA extraction: total RNA containing preserved miRNAs was extracted from 100 μl individual serum using miR-VanaParis extraction kit (AM1556, Ambion, Life Technologies, Carlsbad, Calif.) according to the manufacturer's instructions. In order to monitor extraction efficiency and for the minimization of sample-to-sample variation, i) a synthetic C. elegans miR-39 [3,125 fmoles] (MSY0000010, Qiagen, Venlo, The Netherlands) was added to each sample prior to RNA extraction and ii) a standard serum with a known miR-34a Cq value was processed at the same time of tested samples. The washing steps were then performed using miR-VanaParis washing solutions (8680G & 8543G14 Ambion, Life Technologies, Carlsbad, Calif.) and centrifugation to avoid ethanol carryover. The total RNA including miRNAs was eluted in DNAse/RNAse-free water via centrifugation and immediately stored at −80° C. until use.

Reverse transcription: A fixed volume of 5 μl of total RNA from serum samples or synthetic hsa-miRNA-34a (single strand sequence=5′Phos-UGGCAGUGUCUUAGCUGGUUGU-3′ (SEQ ID NO:1); Integrated DNA Technologies) diluted to 3.125 fmol/mL (used for standard curve construction and miR-34a copies number calculation) were concomitantly reverse transcribed using TaqMan MicroRNA Reverse transcription Kit (4366597, Applied Biosystems, Life Technologies, Carlsbad, Calif.). Reverse transcription reaction was carried out in a final mixture of 15 μL containing 10 μL of TaqMan MicroRNA Assay 5× and incubated in a thermocycler GeneAmp® PCR System 9400 from Applied Biosystem. cDNAs were stored in low binding tubes at −20° C. until further use.

Real-time qPCR: Expression of mature miRNAs was quantified according to the manufacturer's instructions using the Taqman miRNA RT-qPCR Assay 20× and TaqMan Universal Master Mix II, no Uracil-N-Glycosidase (UNG) 2× (Applied Biosystems, Life Technologies, Carlsbad, Calif.). A fixed volume of 5 total RNA was used as a template for the qPCR assay using a CFX96™ Real-Time System. The hsa-miR-34a-5p TaqMan assay was used. The RT product from synthetic miRNAs was serially diluted and PCR was performed on all samples (standards and serum-derived RNA). Standard curve was performed in duplicate and used to convert Cq data in copies/4. The Cq Determination mode was Regression. Transcript abundance is expressed in Cq.

The sequences of mature miRNA and Taq Man assay ID are reported in the following table:

| | | miRbase | Assay |
|---|---|---|---|
| miRNA ID | Sequence | Number | ID |
| cel-miR- | UCACCGGGUGUAAAUCAGCUUG | MIMAT0000010 | 000200 |
| 39-3p | (SEQ ID NO: 2) | | |
| hsa-miR- | UGGCAGUGUCUUAGCUGGUUGU | MIMAT0000255 | 000426 |
| 34a-5p | (SEQ ID NO: 1) | | |

Data used in the construction of the algorithm were in Cq format.

### F. NIS4 Score Calculation

NIS4 was calculated from the following marker levels measured as provided above:
level of hsa-miR-34a-5p in serum;
level of alpha 2 macroglobulin in serum;
level of YKL-40 in serum; and
level of HbA1c in blood EDTA.

The NIS4 score was calculated as provided in application WO2017167934, defined as a logistic function with the serum level of has-miR-34a-5p expressed in Cq unit.

### G. Bioinformatics Analysis

The objective of the analyses is to discover biomarkers that can be related to the identification of NASH patients to be treated. In the present analysis, patients to be treated (TBT) are defined as having the following biopsy-derived parameters:
steatosis score≥1;
hepatocyte ballooning score≥1;
lobular inflammation score≥1;
NAS≥4; and
fibrosis stage≥2.

### Test of Collinearity

Pearson correlation was calculated two by two between quantitative variables. When two variables presented a correlation superior to 0.7 for analysis using plasma miRNA or 0.6 for analysis using serum miRNA, univariate test of difference in their mean in relation to the response variable defining patients TBT were done.

### Bootstrap Model

In the bootstrap modelling process, a logistic generalized linear model of the response variable (defining TBT/NTBT patients) in relation to explanatory variables (biomarkers) is computed on all patients from the overall dataset. A backward variable selection is done and the optimal algorithm is selected using AIC. The significance of variable coefficients from this optimal algorithm is then tested by running the algorithm using 1000 bootstrap samples. Coefficients that show 95% confidence interval excluding zero are considered significant. The algorithm is then validated by calculating ROC, AUC, optimal threshold, total accuracy, sensitivity, specificity, positive predictive value and negative predictive value.

Comparison of Models and Algorithms

Global diagnostic performances of individual biomarkers and multiparametric scores were assessed through Receiver Operating Characteristic (ROC) curve and corresponding area Under-the-Curve (AUROC). AUROC values are provided with 95% CI derived from 1000 bootstrasping of the tested cohort. Statistical differences between ROCs were assessed according to DeLong test (DeLong, 1988).

Diagnostic metrics (total accuracy, sensitivity, specificity, positive predictive value/PPV, negative predictive value/NPV, positive likelihood ratio/LR+ and negative likelihood ratio/LR−) are provided with 95% CI calculated with the asymptotic formula based on the normal approximation to the binomial distribution (Fleiss, 2003).

All statistical analyses were performed using R version 3.4.1 (R Core Team, 2017).

### RESULTS

1. Combination of FIBROSCAN® and NIS4 scores to improve advanced NASH detection. NIS4 score (Table 2).

The NIS4 score had a 72.46% specificity and 60.32% sensitivity. 88.9% of TBT predictions are good. The AUC of this model in this particular population is 0.6637 (0.6039-0.718 with 95% Confidence Interval (C.I.)).

FIBROSCAN® data predicted 25% of healthy patients (FIBROSCAN®<7pPa), which is close to our 21.5% NTBT observed using biopsy data. If we combine FIBROSCAN® score and biopsy data, we can conclude that FIBROSCAN® data are a good predictor of patients with advanced fibrosis but a bad predictor of NTBT patients. The FIBROSCAN® analysis had a 49.28% specificity and 81.75% sensitivity. 85.48% of TBT predictions. The AUC of this model is 0.6551 (0.5912-0.7216 with 95% C.I.).

Descriptive statistical analysis was performed between NIS4 score values and FIBROSCAN® data. No correlation was found. Since no interaction was detected, NIS4 score and FIBROSCAN® data were used as fixed factors in the modeling of a new algorithm combining the NIS4 score and FIBROSCAN® data to differentiate NTBT from TBT patients.

Coefficients were determined for this new algorithm, referred to as NIS4+FS:

$$S 2 ∼ \frac{{e}^{Y 2}}{1 + {e}^{Y 2}}$$

$$Y 2 = 1 + e * S 1 + f * FS$$

wherein:
S1 is the NIS4 score;
FS is the FIBROSCAN® data in kPa;
1 is the constant of the logistic function;
e is a coefficient associated to the NIS4 score; and
f is a coefficient associated to the FIBROSCAN® data.

Particular values of these constant and coefficients are as follows:

1 from −3.6296 to −0.2985,
e from 1.539 to 5.629, and
f from 0.0107 to 0.3229.

The NIS4+FS score had 85.5% specificity and 60.31% sensitivity of the NIS4+FS score. 93.82% of TBT predictions are good. The AUC of this model is 0.7772 (0.7141-0.8316 with 95% C.I.).

Therefore, the specificity of this model is greater than NIS4 score or FIBROSCAN® data alone. The AUC is also significantly better than individual NIS4 score or FIBROSCAN® data.
2. Combination of FIBROSCAN® and NIS4 individual variables to improve advanced NASH detection (Table 2).

A new step wise modeling by logistic regression was performed using A2M, hsa-miR-34a-5p and YKL-40/CHI3L1, HbA1c and FIBROSCAN®. Only 4 parameters were retained: A2M, hsa-miR-34a-5p and YKL-40/CHI3L1 and FIBROSCAN®. HbA1c was eliminated from the model.

The removal of HbA 1 c is interesting since HbA 1 c is quantified in blood samples whereas A2M, hsa-miR-34a-5p and YKL-40/CHI3L1 are determined in serum. In addition, HbA1c determination requires an HPLC.

Coefficients were determined for a new NIS3+FS score:

$$S 3 ∼ \frac{{e}^{Y 3}}{1 + {e}^{Y 3}}$$

$$Y 3 = m + g * A + h * B + i * C + j * FS$$

wherein:
A is the serum level of hsa-miR-34a-5p in Cq;
B is the serum level of alpha 2 macroglobulin in g/L;
C is the serum level of YKL-40 in ng/mL;
FS is the FIBROSCAN® data;
m is the constant of the logistic function;
g is a coefficient associated to the serum level of hsa-miR-34a-5p;
h is a coefficient associated to the serum level of alpha 2 macroglobulin;
i is a coefficient associated to the serum level of YKL-40; and
j is a coefficient associated to the FIBROSCAN® data.

Particular values of these constant and coefficients are as follows:

m from −2.52 to 35.38,
g from −1.2061 to −0.0355,
h from 0.3104 to 1.7716,
i from −0.0015 to 0.0207, and
j from −0.0096 to 0.3465.

The NIS3+FS score had a 85.5% specificity and 61.11% sensitivity. 93.9% of TBT predictions are good. The AUC of this model is 0.8054 (0.7496-0.8514 with 95% C.I.).

Therefore, it was shown that neither the specificity nor the sensitivity was affected by the removal of HbA1c parameter.

Then we tried to remove YKL-40/CHI3L1 from this model.

Coefficients were determined for a new model NIS2+FS score:

$$S 4 ∼ \frac{{e}^{Y 4}}{1 + {e}^{Y 4}}$$

$$Y 4 = n + o * A + p * B + q * FS$$

wherein:
A is the serum level of hsa-miR-34a-5p in Cq;
B is the serum level of alpha 2 macroglobulin in g/L;
FS is the FIBROSCAN® data;
n is the constant of the logistic function;
o is a coefficient associated to the serum level of hsa-miR-34a-5p;
p is a coefficient associated to the serum level of alpha 2 macroglobulin; and
q is a coefficient associated to the FIBROSCAN® data.

Particular values of these constant and coefficients are as follows: n from −9.607 to 35.175,

o from −1.2012 to 0.2127,
p 0.4424 and 1.9381, and
q 0.0258 and 0.3617.

The NIS2+FS score had a 86.95% specificity and 58.73% sensitivity. 94.27% of TBT predictions were good. The AUC of this model is 0.801(0.7412-0.855 with 95% C.I.).

It is herein shown that the specificity of the NIS2+FS model was even greater than the model NIS3+FS. The sensitivity was slightly but not significantly affected by the removal of YKL-40/CHI3L1 parameter.

In conclusion, NIS3+FS and NIS2+FS scores are equivalent in terms of performance.

### SUMMARY OF THE DATA

TABLE 2
Performances of the models with high cut off values to discriminate patients with NAS ≥4 and
F ≥2 from patients with NA <4 or F <2.

| TBT | | | | | | |
|---|---|---|---|---|---|---|
| NAS ≥4 | | | | | | |
| and F ≥2 | | | | | NIS3 + | NIS2 + |
| (S, HB and | | | | NIS4 + | FIBROSCAN ® | FIBROSCAN ® |
| LI) ≥1 | | NIS4 | FIBROSCAN ® | FIBROSCAN ® | MODEL A | MODEL B |
| TBT | TP | 152 | 206 | 152 | 154 | 148 |
| | FN | 100 | 46 | 100 | 98 | 104 |
| NTBT | TN | 50 | 34 | 59 | 59 | 60 |
| | FP | 19 | 35 | 10 | 10 | 9 |
| Accuracy | | 62.93 | 74.77 | 65.73 | 66.35 | 64.79 |
| Sensitivity | | 60.32 | 81.75 | 60.31 | 61.11 | 58.73 |
| Specificity | | 72.46 | 49.28 | 85.51 | 85.51 | 86.95 |
| PPV | | 88.89 | 85.48 | 93.82 | 93.90 | 94.26 |
| NPV | | 33.33 | 42.50 | 37.10 | 37.58 | 36.58 |
| LR+ | | 2.19 | 1.61 | 4.16 | 4.21 | 4.50 |
| LR− | | 0.55 | 0.37 | 0.46 | 0.45 | 0.47 |
| AUC | | 0.6639 | 0.6551 | 0.7772 | 0.8054 | 0.801 |

DeLong E R, DeLong D M, Clarke-Pearson D L. Comparing the areas under two or more correlated receiver operating characteristic curves: a nonparametric approach. Biometrics 1988;44(3):837-45.
Fleiss J L, Levin B, Paik M C. Statistical Methods for Rates and Proportions, Third Edition: John Wiley & Sons, Inc., 2003.

1-14. (canceled)

15. A method for the diagnosis of non-alcoholic steatohepatitis (NASH), for the classification of a subject as a receiver or non-receiver of a treatment for NASH, or for monitoring the efficiency of a treatment for NASH, comprising:
i) measuring liver fibrosis of said subject with a physical method; and
ii) measuring the level of at least one circulating marker in a body fluid of said subject, selected from the group consisting of hsa-miR34a, A2M, YKL40 and Hb1Ac.

16. The method according to claim 15, wherein step i) comprises measuring liver stiffness of said subject.

17. The method according to claim 16, wherein stiffness measure is done by measuring the difference in velocity of elastic shear wave propagation in the liver.

18. The method according to claim 15, comprising the measuring of the level of at least two circulating markers selected in the group consisting of hsa-miR34a, A2M, YKL40 and Hb1Ac.

19. The method according to claim 15, comprising the measuring of the level of hsa-miR34a and A2M.

20. The method according to claim 15, comprising the measuring of the level of at least three circulating markers selected from the group consisting of hsa-miR34a, A2M, YKL40 and Hb1Ac.

21. The method according to claim 20, comprising the measuring of the level of hsa-miR34a, A2M and YKL40.

22. The method according to claim 20, comprising the measuring of the level of hsa-miR34a, A2M, YKL40 and Hb1Ac.

23. The method according to claim 15, wherein measures i) and ii) are combined to calculate a score for the diagnosis of non-alcoholic steatohepatitis (NASH), for the classification of a subject as a receiver or non-receiver of a treatment for NASH, or for monitoring the efficiency of a treatment for NASH.

24. The method according to claim 15, wherein said method is for the classification of a subject potentially having a steatosis score≥1, a hepatocyte ballooning score≥1, a lobular inflammation score≥1, a NAS≥4 and a fibrosis stage≥2.

25. A method for the treatment of NASH comprising classifying a subject according to the method according to claim 15 and treating a subject classified as a receiver of a treatment for NASH with an anti-NASH compound.

26. The method according to claim 25, wherein said anti-NASH compound is elafibranor or a pharmaceutically acceptable salt thereof

27. The method according to claim 25, wherein said anti-NASH compound is nitazoxanide or a pharmaceutically acceptable salt thereof.

28. The method according to claim 25, wherein said anti-NASH compound is a combination of elafibranor or a pharmaceutically acceptable salt thereof and nitazoxanide or a pharmaceutically acceptable salt thereof.
